(19) United States
(12) Patent Application Publication
(10) Pub. No.: US 2014/0025115 A1
(43) Pub. Date: Jan. 23, 2014

(54) Clamping Device of a Flexible Rod for a Minimally Invasive Surgery

(71) Applicant: Chih-Hsuan Wei, Chiayi City (TW)

(72) Inventor: Chih-Hsuan Wei, Chiayi City (TW)

(21) Appl. No.: 13/555,661

(22) Filed: Jul. 23, 2012

(51) Int. Cl. A61B 17/70

(52) U.S. Cl. 606/254

(57) ABSTRACT

A clamping device of a flexible rod for a minimally invasive surgery contains a retaining set and a flexible rod. The retaining set includes a locking member and a clamping seat. The locking member has an inner hexagonal hole defined therein and outer threads formed on an outer rim thereof. The clamping seat has a concaved arcuate face defined on a bottom end thereof and axially connecting with the locking member. The flexible rod is circular and is retained with the concaved arcuate face of the clamping seat.

### BACKGROUND OF THE INVENTION

1. Field of the Invention

The present invention relates to a clamping device of a flexible rod for a minimally invasive surgery which has a flexible clamping seat to protect the flexible rod by which a plurality of bone screws are limited.

2. Description of the Prior Art

A conventional clamping device for positioning a bone screw is used to retain the bone screw with a connecting rod in a surgery, but the bone screw can not operate flexibly, and the connecting rod can not be protected well. Furthermore, the conventional clamping device still has the following defects:

1. The conventional clamping device is made of hard material, so the at least one bone screw can not bend and rotate flexibly.

2. No any flexible component is used as a buffer as screwing the at least one bone screw with the connecting rod, so the connecting rod is worn easily and will hurt a patient.

The present invention has arisen to mitigate and/or obviate the afore-described disadvantages.

### SUMMARY OF THE INVENTION

The primary object of the present invention is to provide a clamping device of a flexible rod for a minimally invasive surgery which has a flexible rod to make at least one bone screw operate flexibly with a spine so as to further recover a bend and rotation flexibility after implanting the at least one bone screw.

Secondary object of the present invention is to provide a clamping device of a flexible rod for a minimally invasive surgery in which the flexible rod is made of flexible material so as to prevent the flexible rod from damage and to prevent the flexible rod from hurting a patient, thus screwing the at least one bone screw with the spine.

Further object of the present invention is to provide a clamping device of a flexible rod for a minimally invasive surgery which matches with at least one elongated bone screw well to minimize a cut wound in the minimally invasive surgery.

Further object of the present invention is to provide a clamping device of a flexible rod for a minimally invasive surgery in which the flexible rod is made of different flexible materials based on the patient's age, thus increasing use flexibility.

A clamping device of a flexible rod for a minimally invasive surgery in accordance with a preferred embodiment of the present invention contains: a retaining set and a flexible rod, wherein

the retaining set includes a locking member and a clamping seat, the locking member has an inner hexagonal hole defined therein and outer threads formed on an outer rim thereof, the clamping seat has a concaved arcuate face defined on a bottom end thereof and axially connecting with the locking member, the locking member is made of metal material, and the clamping seat is made of plastic, rubber, high-density foam, or silicone;

the flexible rod is circular and is retained with the concaved arcuate face of the clamping seat and made of plastic, rubber, high-density foam, or silicone.

### BRIEF DESCRIPTION OF THE DRAWINGS

FIG. 1 is a perspective view showing the exploded components of a clamping device of a flexible rod for a minimally invasive surgery according to a preferred embodiment of the present invention.

FIG. 2 is a cross sectional view showing the assembly of the clamping device of the flexible rod for the minimally invasive surgery according to the preferred embodiment of the present invention.

FIG. 3 is a perspective view showing the application of the clamping device of the flexible rod for the minimally invasive surgery according to the preferred embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

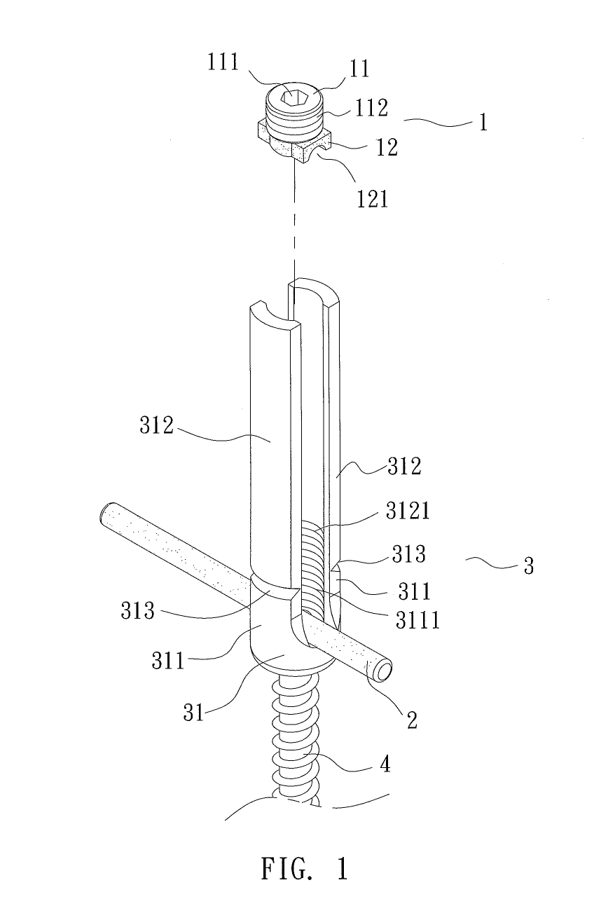

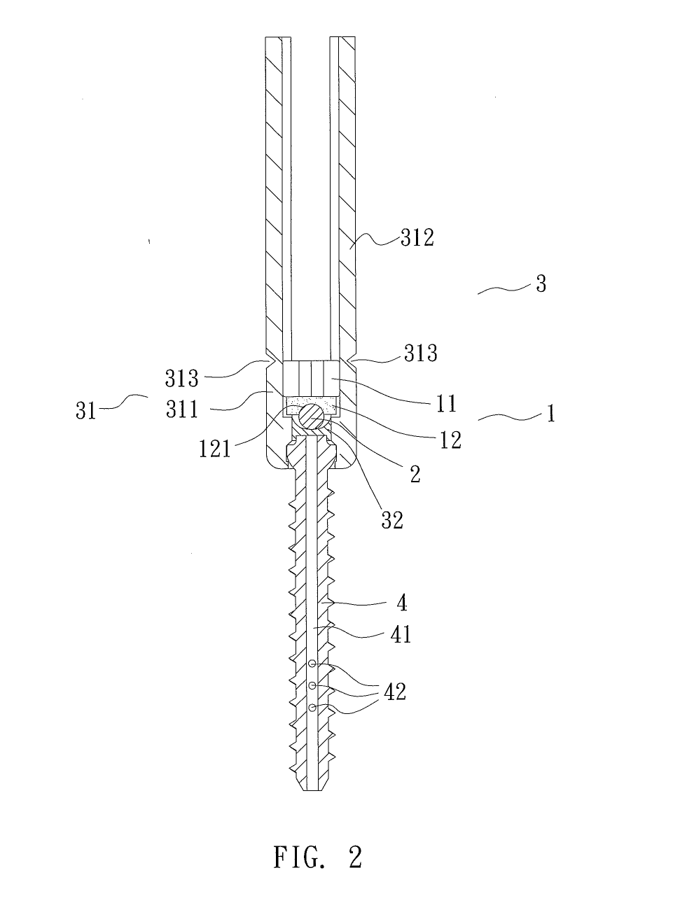

Referring to FIGS. 1 and 2, a clamping device of a flexible rod for a minimally invasive surgery according to a preferred embodiment of the present invention comprises: a retaining set 1 and a flexible rod 2, wherein

the retaining set 1 includes a locking member 11 and a clamping seat 12, the locking member 11 has an inner hexagonal hole 111 defined therein and outer threads 112 formed on an outer rim thereof, the clamping seat 12 has a concaved arcuate face 121 defined on a bottom end thereof and axially connecting with the locking member 11, the locking member 11 is made of metal material, and the clamping seat 12 is made of plastic, rubber, high-density foam, or silicone;

the flexible rod 2 is circular and is retained with the concaved arcuate face 121 of the clamping seat 1,2 and made of plastic, rubber, high-density foam, or silicone.

In operation, the flexible rod 2 is slid between two first side walls 311 of a holder 31 of a bottom of a body 3 from two second side walls 312 of the body 3 of a bone screw as illustrated in FIG. 1. Thereafter, the retaining set 1 is placed into the body 3 by ways of the clamping seat 12 along the two second side walls 312 of the body 3 of the screw bone until the outer threads 112 of the locking member 11 of the retaining set 1 contact with second inner threads 3121 of the two second side walls 312 of the body 3. In the meantime, the retaining set 1 can not slide downward anymore, so a tool (such as a hexagon wrench) is retained with the inner hexagonal hole 111 to rotate the locking member 11 so that the outer threads 112 screw with the second inner threads 3121 and then screw with first inner threads 3111 of the two first side walls 311. At this moment, the locking member 11 and the clamping seat 12 are in a releasably axial connecting state, i.e., the clamping seat 12 slides downward along the two second side walls 312 and the two first side walls 311 but does not rotate with the locking member 11 until the concaved arcuate face 121 of the clamping seat 12 contacts with the flexible rod 2, as shown in FIG. 2, thereafter the locking member 11 is rotated further so that the concaved arcuate face 121 of the clamping seat 12 presses the flexible rod 2, and the flexible rod 2 is fixed between the clamping seat 12 and an axial pedestal 32, such that the retaining set 1 enters into the two first side walls 311 of the holder 31 completely, and an upper rim of the locking member 11 flushes with a V-shaped slot 313. Furthermore, a screw 4 includes a plurality of orifices 42 communicating with an aperture 41.

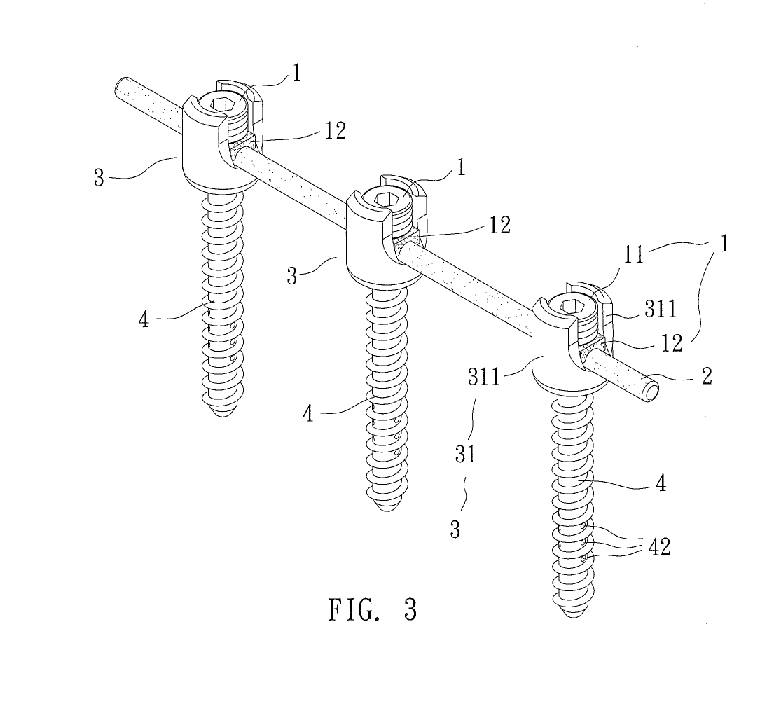

Referring further to FIG. 3, after the operation as shown in FIG. 3, locking members 11 of a plurality of retaining sets 1 are screwed into bodies 3 of a plurality of bone screws and are located between first side walls 311 of a plurality of holders 31, the flexible rod 2 is retained by a plurality of clamping seats 12 securely so that a plurality of screws 4, each having a plurality of orifices 42, screw with a spine.

Thereby, in a minimally invasive surgery, the clamping device of the present invention screws the plurality of screws 4 with the spine precisely, and the plurality of bone screws are limited by the plurality of clamping seats 12 and the flexible rod 2 so that only a little wound is cut in the minimally invasive surgery to lower infection risk and to recover quickly. As shown in FIG. 2, the V-shaped slot 313 defined between the two first side walls 311 and the two second side walls 312 is used as a forcing position of an auxiliary tool to remove the two second side walls 312 after implanting the bone screw, so only the body 3 with the two first side walls 311 is left in a patient's body.

Therefore, the clamping device of the present invention has the retaining set 1 to retain the flexible rod 2 securely by rotating locking member 11, the clamping seat 12 protects the flexible rod 2, and the flexible rod 2 limits the bodies 3 of the plurality of bone screws so that the spine has a bend and rotation flexibility after implanting the plurality of bone screws and a cut wound in the minimally invasive surgery is minimized.

While we have shown and described various embodiments in accordance with the present invention, it is clear to those skilled in the art that further embodiments may be made without departing from the scope of the present invention.

1. A clamping device for a minimally invasive surgery comprises:
a retaining set including a locking member and a clamping seat, with the locking member having an inner hexagonal hole defined in a top end thereof and outer threads formed on an outer rim thereof, with the clamping seat having a concaved arcuate face defined on a bottom end thereof and axially connecting with the locking member;
a body including a holder connecting with a screw, with the holder including two circumferentially and radially spaced side walls to define an inner passage and first and second slots, with the inner passage defined by the sidewalls including inner threads, with the outer threads of the locking member threadably received in the inner threads;
an axial pedestal received in the inner passage and having a concaved arcuate face defined on a top end thereof; and
a flexible rod being circular, with the flexible rod retained in the first and second slots and abutting with the concaved arcuate faces of the clamping seat and the axial pedestal and with the V-shaped slot of each of the two sidewalls being flush with the top end of the locking member, with the two sidewalls opposite the V-shaped slot from the screw being removable after implanting the body, the locking member, the clamping seat, and the axial pedestal.

2. The clamping device for the minimally invasive surgery as claimed in claim 1, wherein the locking member is made of metal material.

3. The clamping device for the minimally invasive surgery as claimed in claim 1, wherein the clamping seat is made of plastic.

4. The clamping device for the minimally invasive surgery as claimed in claim 1, wherein the clamping seat is made of rubber.

5. The clamping device rod for the minimally invasive surgery as claimed in claim 1, wherein the clamping seat is made of high-density foam.

6. The clamping device for the minimally invasive surgery as claimed in claim 1, wherein the clamping seat is made of silicone.

7. The clamping device for the minimally invasive surgery as claimed in claim 1, wherein the flexible rod is made of plastic

8. The clamping device for the minimally invasive surgery as claimed in claim 1, wherein the flexible rod is made of rubber.

9. The clamping device for the minimally invasive surgery as claimed in claim 1, wherein the flexible rod is made of high-density foam.

10. The clamping device for the minimally invasive surgery as claimed in claim 1, wherein the flexible rod is made of silicone.
